(19) United States
(12) Patent Application Publication
(10) Pub. No.: US 2012/0310197 A1
(43) Pub. Date: Dec. 6, 2012

(54) Absorbent Article Having A Troughed Film As A Transfer Layer

(76) Inventor: Paul Eugene Thomas, (US)

(21) Appl. No.: 13/487,584

(22) Filed: Jun. 4, 2012

Related U.S. Application Data
(60) Provisional application No. 61/492,846, filed on Jun. 3, 2011.

(51) Int. Cl. A61F 13/51; B28B 21/36

(52) U.S. Cl. 604/378; 425/388

(57) ABSTRACT

The application relates to absorbent articles and in particular to absorbent articles containing a transfer layer having a three-dimensional structure that is orientated for improved directional flow of bodily fluids and distribution within the absorbent article.

### CROSS REFERENCE TO RELATED APPLICATIONS

This application claims the benefit of U.S. Provisional Application No. 61/492846, filed Jun. 3, 2011.

### BACKGROUND OF THE DISCLOSURE

The application relates to absorbent articles and in particular to absorbent articles containing a transfer layer having a three-dimensional structure that is orientated for improved directional flow of bodily fluids and distribution within the absorbent article.

A transfer layer, which is also known in the art as an acquisition distribution layer or “ADL”, has been used in absorbent articles. Both nonwoven webs and three-dimensional formed films have found use as transfer layer in the past. A transfer layer is typically positioned between the topsheet and the absorbent core of an absorbent article. Transfer layers are used to provide void volume, which serves as a temporary reservoir to collect and hold bodily fluids until the fluids can be absorbed by the absorbent core of the absorbent article. Transfer layers have been employed to promote lateral flow of fluids in a direction generally parallel to the plane of the transfer layer, thereby permitting more surface area of the absorbent core to be used to absorb the bodily fluids such as that discussed in U.S. Pat. No. 7,378,568, U.S. Pat. No. 6,700,036 and U.S. Pat. No. 6,610,904. Transfer layers are also used to improve comfort by reducing rewetting or evacuation of the bodily fluids contained in the absorbent core to the users' skin.

It is customary today for high absorbency gel-type material to be used in the absorbent core of the absorbent article. However, high absorbency gel-type materials are relatively slow at the uptake of bodily fluids, resulting in unabsorbed or free fluid in the absorbent article which can increase the risk of leakage and user discomfort. There is a need for improved transfer layers providing improved directional flow down the length of the article to promote increased speed and amount of absorption and the uniform distribution of fluids over the absorbent core, prevention of leg cuff leakage, providing a cooler comfort for the wearer, and reduction of surface wetness in the topsheet while reducing or eliminating rewet of the absorbent article.

### SUMMARY OF THE DISCLOSURE

The present application relates to an absorbent article comprising a width and a length, the length running from a front side edge of the article, through a crotch area, to a back side edge of the article, the width of the article is perpendicular to the length and runs from a left side edge of the article to the right side edge of the article; the absorbent article further comprises a topsheet, a backsheet, an absorbent core positioned between the topsheet and the backsheet, and a transfer layer positioned between the topsheet and the backsheet in the crotch area; the transfer layer comprises a primary plane, a secondary plane and a third plane; extended from primary plane to the secondary plane are a plurality of troughs; extending from primary plane to the third plane are a plurality of protrusions; the protrusions comprise an apertures in the primary plane, sidewalls and a terminal end comprising an aperture, the terminal end and the aperture are located in the third plane; wherein the third plane is in contact with the topsheet and the plurality of ridges are orientated parallel to the length of the absorbent article.

The present application also relates to a cylindrical vacuum forming screen comprising a base pattern of apertures and one or more shallow spiral grooves encompassing one or more wires affixed around the circumference of the forming screen.

### BRIEF DESCRIPTION OF THE FIGURES

FIG. 1 is a perspective view of an absorbent article having a transfer layer web.

FIG. 2 cross-sectional view of the absorbent article, as seen along lines and arrows II-II of FIG. 1.

FIG. 3 is a magnified sectional plan view of a transfer layer cross-sectional view from the female surface perspective.

FIG. 4 is an SEM photograph of an embodiment of the transfer layer, as seen in cross-section and showing the sinusoidal structure of the transfer layer.

FIG. 5 is an SEM photograph of an embodiment of the transfer layer, as seen from the male surface perspective of the transfer layer.

FIG. 6 is a perspective view of a cylindrical vacuum forming screen of the present application, not to scale.

FIG. 7 schematic representation of a diaper used as a comparison to measure fluid flows, as seen in cross-section.

FIG. 8 schematic representation of a test specimen comprising the transfer layer to measure fluid flows, as seen in cross-section.

FIG. 9 is a schematic representation of a test apparatus used to measure fluid flows, as seen in plan.

FIG. 10 is a side elevation view of the test apparatus of FIG. 9.

FIG. 11 is a plan view of the test apparatus of FIG. 9 and the segmenting of the test specimen to test for liquid distributions.

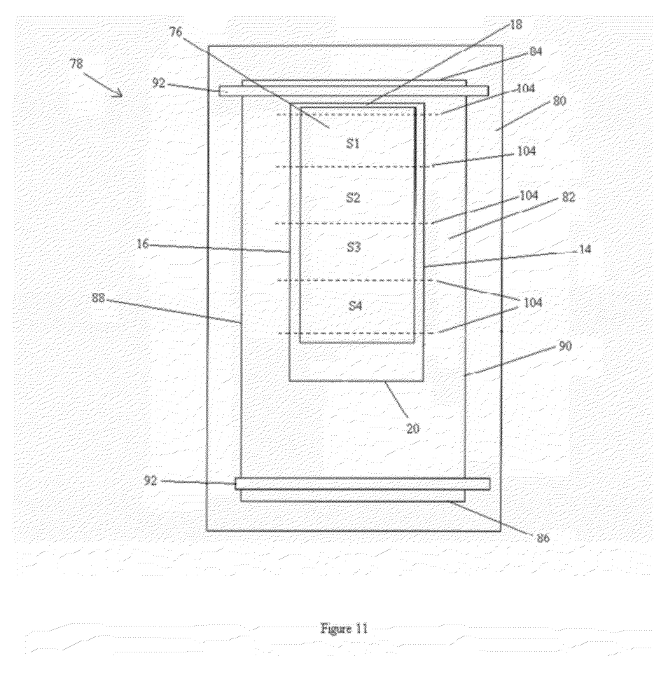

### DETAILED DESCRIPTION OF THE EMBODIMENTS

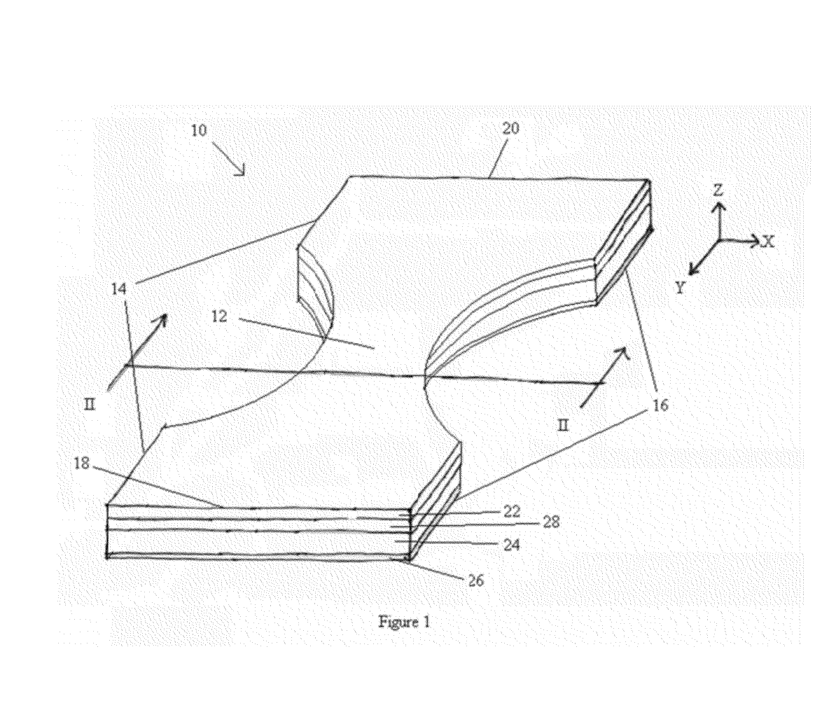

Absorbent articles 10, such as diapers, generally have a width and a length, sometimes also referred to as a longitudinal axis and lateral axis, respectively. The length (longitudinal axis) of the article is the dimension or direction running from the front side edge 18 of the article, through the crotch area 12, to the back side edge 20 of the article. The width (lateral axis) of the article is the dimension or direction perpendicular to the longitudinal axis and runs from the left side edge 14 of the article to the right side edge 16 of the article. Herein, the longitudinal axis or length of the article is designated as the “Y” direction, the width or lateral axis as the “X” direction, and thickness or depth of the article as the “Z” direction, as illustrated in FIG. 1.

In absorbent articles that are worn between the legs, the X-direction of the absorbent article 10 is generally smaller than the Y-direction, particularly in the crotch area 12. This means that there is less absorbent core 24 in the X-direction as compared to the Y-direction in the crotch area 12 of the absorbent article 10. With less absorbent core 24, the article has less capacity to absorb fluids in the X-direction as compared to the Y-direction. Furthermore, the distance between the point of insult and a left side edge 14, and a right side edge 16 of the article is less than the distance between the point of insult in the crotch area 12 and a front side edge 18, and a back side edge 20 of the article. Accordingly, in current absorbent articles, fluids from an insult have a greater chance of reaching the side edges 14, 16, before being absorbed as compared to reaching a front side edge 18 or a back side edge 20. For these reasons, the absorbent articles are generally more likely to leak along the side edges 14, 16, as opposed to the front and back edges 18, 20.

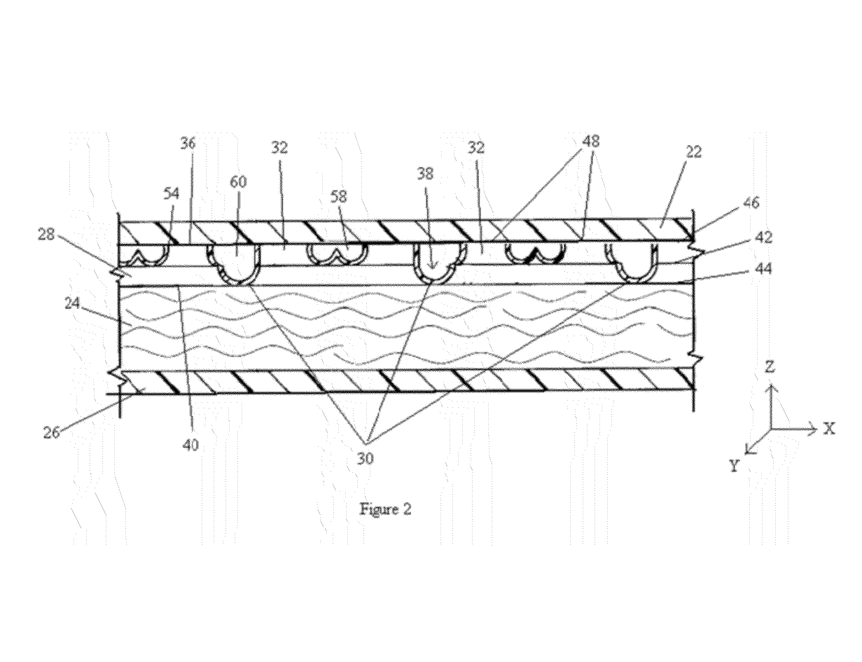

With reference to FIGS. 1 and 2, absorbent articles 10 comprise a topsheet 22, a backsheet 26, an absorbent core 24 positioned between topsheet 22 and backsheet 26, and a transfer layer 28. In accordance with the embodiments, the transfer layer 28 is located between the topsheet 22 and the absorbent core 24. In some embodiments, the transfer layer 28 may be positioned between the backsheet 26 and the absorbent core 24. While the transfer layer 28 may be coextensive with the topsheet 22 and the absorbent core 24 in terms of its length and width, in most absorbent articles this is not necessary. Instead, it is generally sufficient to place the transfer layer 28 in the area of the absorbent article where the insult will occur; i.e., the crotch area 12 or in the areas which will be adjacent to the urethra, the anus and/or the vaginal opening of the user during use of the absorbent article.

The present invention relates to the use of a transfer layer 28 comprising a plurality of troughs 38 and protrusions 32, with the troughs 38 positioned against the absorbent core 24. When an insult occurs, the bodily liquids will pass through the topsheet 22 and contact the third plane 46 of the transfer layer 34. Some of the fluids will enter the apertures in the third plane 46 and flow through the protrusions 32 to the absorbent core 24 under the influence of gravity. The majority of the liquids, however, will enter the troughs 38 where the liquids will be distributed to other areas in the article. As the troughs 38 fill up, the liquids begin to flow into the apertures 36 in the third plane 46 and down through the protrusions 32 to the absorbent core 24. While not intending to be bound by any particular theory, the spillage of the liquids into the protrusions 32 is believed to create a siphoning action which maintains a consistent flow of liquids from the trough 38 through the protrusions 32 to the absorbent core 24.

In light of the mechanism of action just described, the transfer layer 28 provides multiple benefits. First, it directs bodily fluids to areas outside the primary insult area, which maximizes utilization of the absorbent capacity of the absorbent core 24. Second, the troughs 38 provide pathways for vapors to traverse the absorbent article, which decreases the local humidity beneath the topsheet 22 within the article and promotes user comfort and health. Third, the orientation of the troughs 38 along the length of the transfer layer 28 in the Y-direction of the absorbent article effectively prevent liquids from flowing in the X-direction, which would eliminate or greatly reduce the change of leakage from the leg openings and side edges 14, 16 of the article.

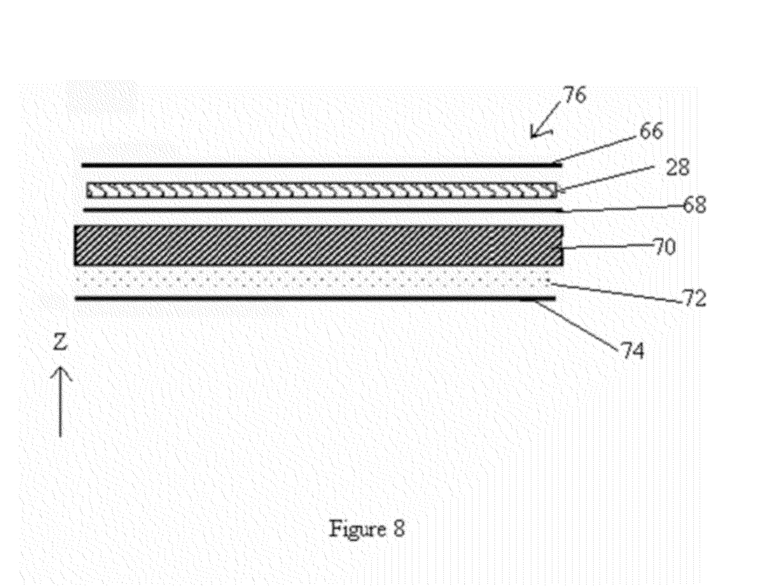

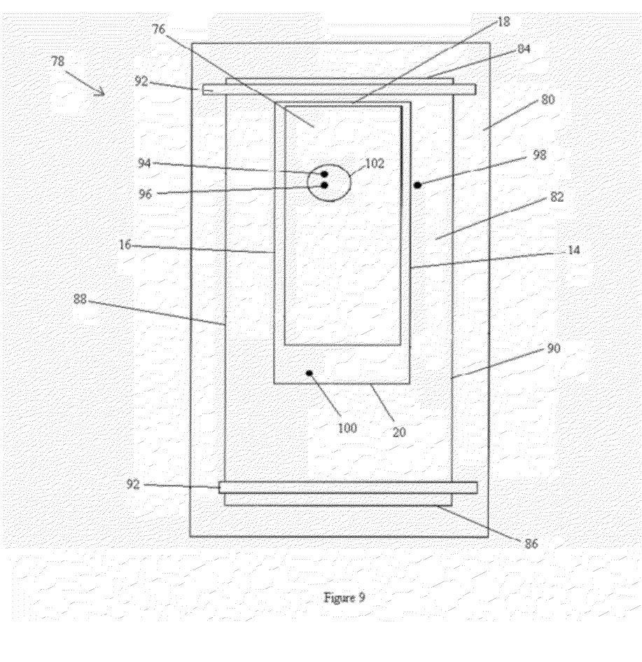

The orientation of the transfer layer 28 with the third plane 46 of the transfer layer 34 facing the topsheet 22 is contrary to the more typical orientation of the acquisition distribution layer as discussed in U.S. Pat. No. 7,378,568 (at Col. 8, line 47—Col. 9, line 53; Col. 12, lines 36-57; FIGS. 8 and 9) with the male side oriented toward the absorbent core. While the more typical orientation of the female side of the acquisition distribution layer facing the topsheet provides for good rewet properties, it will be shown below that distribution in the absorbent core 24 is improved with the orientation of the transfer layer 28 with third plane 46 of the transfer layer 28 facing the topsheet 22.

The transfer layer 28 may function to control rewet, a phenomenon whereby unabsorbed or “free” fluid within the article is present on the surface of the topsheet 22. Rewet is comprised of a surface wetness component and a back wetting component. Surface wetness refers to liquids that remain on the surface of the topsheet 22 after an insult. Back wetting refers to fluids that have once passed through the topsheet 22 but transfer back to the topsheet 22 surface. Back wetting is generally more pronounced when the article is under load or compression, whereby fluids are forced back through the topsheet 22. The compression can occur, for example, when an infant urinates in the diaper and then sits.

The transfer layer 28 controls the rewet by providing a physical barrier to back wetting. In certain situations, transfer layer 28 can also reduce surface wetness on the topsheet 22 by facilitating transfer of stationary fluids that would otherwise tend to remain on the topsheet 22.

The transfer layer 28 comprises a Y-direction (machine direction), a X-direction (cross direction), and a Z-direction (thickness). The Y-direction is defined by the direction in which the film made into the transfer layer 28 passes through the manufacturing process. Perpendicular to the Y-direction is the X-direction or cross direction (width) of the transfer layer 28. The thickness of the transfer layer 28 (sometimes also referred to as loft or caliper of the transfer layer 28) is measured in the Z-direction.

As seen in FIG. 2, the transfer layer 28 comprises a primary plane 42 and a plurality of generally linear troughs 38 extending from the primary plane 43 in the Z-direction as viewed from the male surface 34 perspective of the transfer layer 28, as viewed from the female surface 40 perspective, the troughs 38 “become” ridges 30. These troughs 38 extend away from the primary plane 42 of the transfer layer 28 to a height, such that the lowest point of the troughs 38 form a secondary plane 44 (as viewed from the female surface 40, the highest point of the ridges 30 form a secondary plane 44). The height of the troughs 38 is the distance between the primary plane 42 and the secondary plane 44.

The transfer layer 28 also comprises a plurality of protrusions 32 originating on the primary plane 42 comprise sidewalls 54 and protrude outwardly in the Z-direction in an opposite Z-direction from the secondary plane 44 of the transfer layer 28, the protrusion 32 sidewalls 54 forming a terminal end 48, the terminal end 48 comprising an aperture 36. The protrusions 32 extend from the primary plane 42 of the transfer layer 28 (as viewed from the male surface 34 perspective), the terminal ends 48 forming a third plane 46 (as viewed from the female surface 40 perspective, the protrusions 32 form tapered frustum structures (truncated cones), with the terminal end 48 forming an aperture 36). The distance that the protrusions 32 sidewalls 54 extend away from the primary plane 42 to the third plane 46 is the height of the protrusions 32 and the height is greater than the nominal thickness of the transfer layer 28. The “loft” or “caliper” of the transfer layer 28 is defined as the overall Z-direction dimension of the transfer layer 28, from the lowest point of the trough 38 (secondary plane 44) to the terminal end 48 of the protrusions 32 (third plane 46).

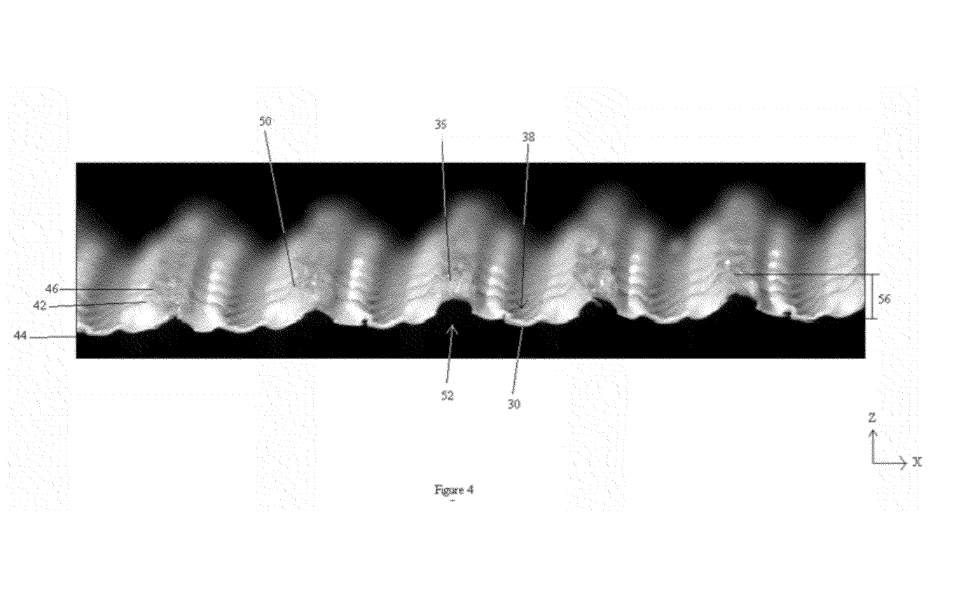

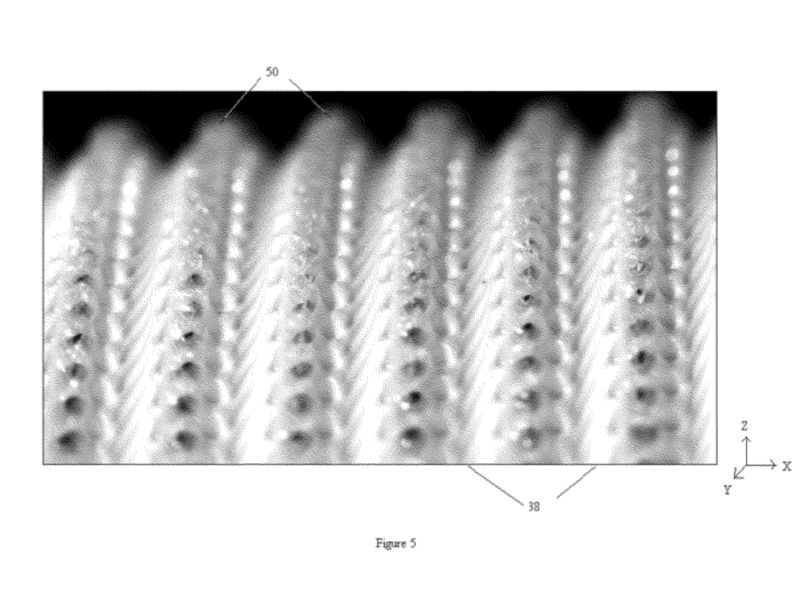

In an embodiment, the transfer layer 28 is a three-dimensional vacuum formed apertured film having a sinusoidal curvilinear shape in cross section as can be seen in FIG. 4, and comprising an alternating series of linear extending peaks 50 and troughs 38 that are adjacent to one another in the X-direction of the transfer layer 28 and extending linearly in the Y-direction of the transfer layer 28. The transfer layer 28 further containing a plurality of hollow protrusions 32 originating on the primary plane 42 of the transfer layer 28 and extending outwardly away from the primary plane 42 and terminating in an aperture in the third plane 36, the protrusions 32 being located on the peaks 50 and extending in the same direction as the peaks 50 in the Z-direction. FIG. 5 shows the transfer layer 28 of FIG. 4 from the male surface 34 perspective with the terminal ends 48 of the protrusions 32 forming the third plane 44 and the troughs 38 forming the secondary plane 44.

With reference to FIGS. 2-5, transfer layer 28 has a plurality of protrusions 32 that originate from the primary plane 42 and protrude upwardly in a Z-direction as viewed from the male surface 34 perspective of the transfer layer 28 (protrude downwardly in a Z-direction as viewed from the female surface 40 perspective of the transfer layer 28). The protrusions 32, as seen in the illustrated embodiments, are in the shape of a truncated cone and are hollow structures defined by an aperture 52 in the primary plane 42 having sidewalls 54 extending therefrom which taper inward and terminate in an aperture 36 in the third plane 46. The aperture 36 corresponds to the aperture of a forming screen as further discussed below. As the protrusion 32 comprises a tapered sidewall 54 structure, the aperture of the third plane 36 is smaller in diameter than the aperture of the primary plane 42 of the transfer layer 28.

The transfer layer 28 further has a plurality of troughs 38 as viewed from the male surface 34 perspective or ridges 30 as viewed from the female surface 40 perspective. In a preferred embodiment, the troughs 38 are linear and have a length that is coextensive with the length of the transfer layer in the Y direction of the transfer layer 28. However, in other embodiments the trough 38 may be of a finite length that is less than the length of the transfer layer in the Y direction of the transfer layer 28. The troughs 38 are preferably oriented in a spaced-apart parallel arrangement as seen in the FIGS. 2-6. However, it is understood that the troughs 38 may be oriented on converging, diverging and/or intersecting paths. Moreover it should be understood that the spacing between each trough 38 need not be consistent across the transfer layer 28. In other words, spacing between adjacent troughs 38 can be the same across the transfer layer 28 in some embodiments or varied spacing between adjacent troughs 38 across the transfer layer 28. In the embodiments, the troughs 38 would be oriented to run parallel in the Y-direction of the article 10. In this embodiment, liquids will be directed to the areas of the article 10 that have more material in the absorbent core 24 and also will restrict movement of liquids toward the side edges (14, 16) of the article, thus reducing leakage.

With reference to FIG. 4, it can be seen that the troughs 38 of the transfer layer 28 provides fluid channels 56, the fluid channels 56 comprising the height of the troughs 38 (ridges 30) and the height of the protrusions 32, or approximately the total loft of the transfer layer 28. Therefore, orienting the transfer layer 28 with the third plane 46 toward the topsheet 22 provides for far greater fluid and vapor handling properties, and a significant barrier to fluids moving in the X-direction compared to U.S. Pat. No. 7,378,568, which positions the acquisition distribution layer with the female side toward the topsheet.

As seen in FIG. 2, the third plane 46 of the transfer layer 28 is preferably maintained in close contact with the topsheet 22 while the secondary plane 44 (troughs 38) is maintained in close contact with the absorbent core 24. To ensure such close contact, the transfer layer 28 may be secured to the topsheet 22, the absorbent core 24, or both using a suitable adhesive. The area between the protrusions 32 and the topsheet 22, as well as the area between the troughs 38 and the topsheet 22 are void spaces 58, 60, respectively. The void spaces 58, 60 are negative space, which means the space it is empty and/or generally free of any fibers, filler, or other materials. In such embodiments, the void spaces 58, 60 provide for substantially unencumbered lateral spillage of liquid and convective flow of vapors.

The troughs 38 and protrusions 32 in the transfer layer 28 may be produced in an embossing process, a hydroforming process, or a vacuum forming process, for example. A preferred process is hydroforming or vacuum forming processes. The size, spacing and other physical properties of the troughs 38 and protrusions 32 are based upon the particular apparatus used to create the transfer layer 28. For example, in a vacuum forming process, a hydroforming process, and some mechanical processes, the film used to form the transfer layer 28 conforms to the shape of an underlying forming screen 106. Accordingly, in such processes, the size, shape and spacing of the troughs 38 and protrusions 32 (or apertures 36) is determined by the size, shape and spacing of the apertures 108 in the forming screen 106 that supports the film while the film is subjected to vacuum pressure, pressurized water streams, or mechanical perforation devices such as pins. See, for example U.S. Pat. No. 4,456,570 and U.S. Pat. No. 3,929,135. The apertures 108 of the forming screen 106 correspond to the apertures 36 and apertures 52 of the transfer layer 28.

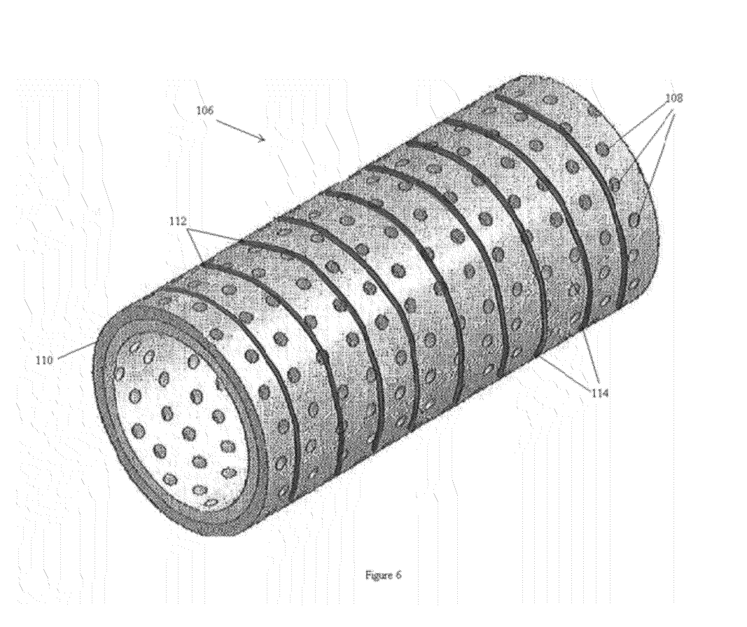

The troughs 38 of the transfer layer 28 may be formed from a film that is brought into contact with a forming screen 106, as exemplified in FIG. 6, having a base pattern 110 that comprises a structure which is a negative or opposite structure as that desired for the trough 38 structure of the transfer layer 28. One embodiment for creating a forming screen 106 having a negative structure for the trough 38 is by affixing a wire 112 around the circumference of a cylindrical vacuum forming screen 106 or by forming an elongated ridge upon a vacuum formed screen 106 and passing a film over the screen 106 in a manner known in the art.

It is a preferred embodiment that a shallow spiral groove 114 is cut in the base pattern 110 of the forming screen 106 encompassing the wire 112 to hold the wire 112 in place. Spiral grooves 114 are cut by lathes by selecting a Thread Per Inch (TPI) setting typically used on lathes for cutting threads in bolts. The resulting height of the troughs 38/ridges 30 is approximately 70% of the diameter of the wire 112 used in the forming screen 106 to make the transfer layer 28. A TPI from 20 to 6 may be utilized for the spiral groove 114 in the base pattern 110 of the forming screen 106. The wire 112 diameters utilized with the spiral groove 114 may be from 0.305 mm to 2.362, such as 1.168 mm. US2005/0003152 at [0049]-[0052] further discusses the attachment of the wire 112 to the forming screen structure 106. Methods described in co-pending US Patent Publication. No.20100151191, incorporated herein by reference, could also be used to advantage for formation of the base pattern 110.

The diameter of the wire 112 selected to be used for the forming screen 106 is related to the diameter of the apertures 108 formed in the base pattern 110 of the forming screen 106. A large diameter aperture 108 as well as a large diameter wire 112 will result in elimination of apertures 108 in the forming screen 106 and therefore result in fewer protrusions 32 in the transfer layer 28. Accordingly, it is desired to use lower TPI values when large diameter apertures 108 are used to provide sufficient space between wires 112 to expose an adequate number of apertures 108 in the forming screen 106 and therefore the resulting number of apertures (36, 52) in the transfer layer 28. Similarly, finer mesh sizes, that is, a larger number of apertures per unit area, will result in a transfer layer 28 with smaller apertures 36 and less loft and thus may be preferred when a thinner absorbent article is preferred. Because the apertures of the corresponding forming screen are smaller, a smaller diameter wire and higher TPI will also be used to create negative structure in the forming screen to result in the linear troughs 38 in order to maintain a low loft of the transfer layer 28.

The number of protrusions 32 aligned per linear inch of the transfer layer 28 is referred to as “mesh count.” The mesh count may range from 2 to 35 or more preferably from 4 to 15, preferably a mesh count of 8.75. It is understood that all numbers within such ranges are included, such that the mesh count can be between 3 and 5, between 4 and 7, between 10 and 15, between 9 and 12, etc.

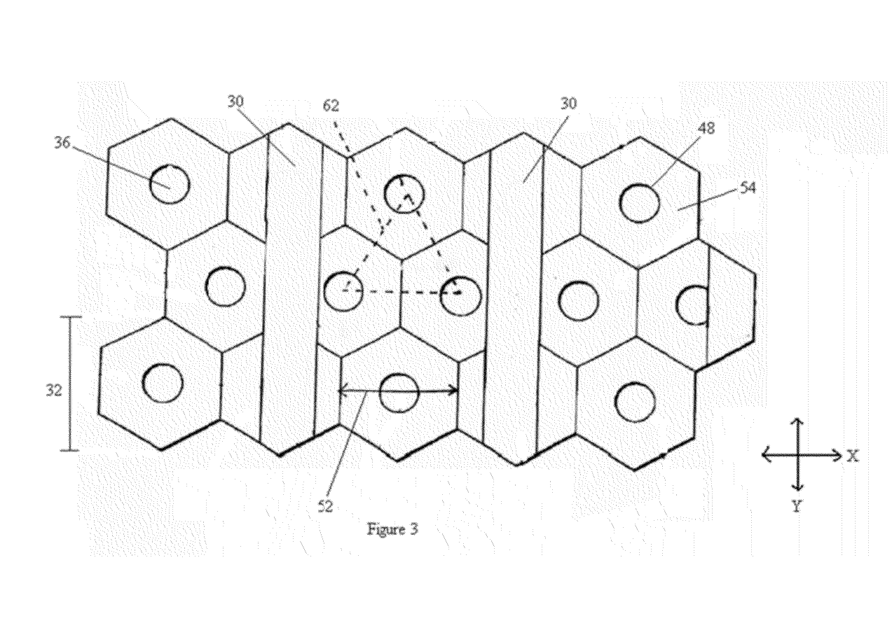

In the embodiment shown, as best seen in FIG. 3, the protrusions 32 have a hexagonal shape when viewed from the female surface 40 of the transfer layer 28. Although a hexagonal pattern is discussed for purposes of illustration, it should be understood that other patterns may also be used for any of the transfer layers 28 discussed herein. Examples of other patterns include circular, oval, elliptical, polygonal, crescent shaped, cat-eye shaped, boat shaped, etc.

The edge-to-edge dimension defining the aperture in the primary plane 52 of the transfer layer 28 can be calculated by dividing the mesh count into one lineal inch. For example, with a mesh count of 8.75, the dimension of the aperture in the primary plane 42 (indicated by 52 in FIG. 3) is calculated as 1÷8.75 or 2.9 mm (0.114 in). The protrusion 32 from the primary plane 42 to the third plane 46, the protrusion 32 typically tapering and rounding to an apex forming the aperture in the third plane 46. The diameter of the aperture 36 is typically about 47% of the dimension of the aperture 52. In such an embodiment, the diameter of the aperture 36 would be 2.9 mm×0.47=1.36 mm (0.0536 in). For nested hexagon patterns, this rule will generally follow for all ranges of mesh counts. Other patterns, such as circular patterns, ovals, ellipses and other polygons will follow slightly different relationships as guided by geometrical factors.

The transfer layers 28 must have sufficient open area to allow for fluid transfer through the apertures in the third plane 36 from the male surface 34 through the protrusions 32, through the apertures 52 and to the absorbent core 24. Open area is a function of the number of apertures in the third plane 36 per unit area (i.e., mesh count) and the diameter of the aperture 36. With the pattern shown in FIG. 3, apertures in the third plane 36 (and the apertures in the primary plane 52) are aligned in a 60° equilateral triangular array, illustrated in FIG. 3 as triangle 62, which is a particularly preferred arrangement for the apertures in the third plane 36 (and the apertures in the primary plane 52).

When using the preferred 60° equilateral triangle array, the open area of the transfer layer 28 can be calculated by the equation:

OA=π(A/2)²

where OA=open area and A=diameter of the aperture 36. By way of example using the nested hexagon pattern of FIG. 3, the diameter of the aperture 52 in the primary plane is 0.114 in (2.9 mm) and the diameter of the aperture in the third plane 36 is 0.0536 inch (1.36 mm). Thus, the open area is calculated to be OA=π(0.0536/2)²=0.002256 in².

The mesh count of the transfer layer 28 is 8.75 protrusions per lineal inch in the X-direction. In a 60° equilateral triangular array, alternate rows of protrusions are offset from one another as seen in FIG. 3. Thus, the mesh count in the Y-direction is 1.15 times the mesh count in the X-direction, which in this particular embodiment is (8.75)×(1.15)=10. The number of protrusions in one square inch of film is then determined by multiplying the mesh count in the X-direction by the mesh count in the Y-direction.

In the embodiment shown in FIG. 3, the number of protrusions is 10×8.75, or 87.5 protrusions per square inch of film. Each protrusion 32 has an aperture 36 which, as calculated above, has an open area of 0.002256 in². Multiplying the number of protrusions per square inch by the open area of the apertures in the third plane 36 (0.002256×87.5) provides a total open area of 0.197 in² per square inch of the transfer layer 28. Most commonly, this is expressed as a percentage open area by multiplying by 100%, so 0.197 in² open area would be expressed as 19.7% open area.

The above calculation of total open area of the transfer layer 28 assumes that the array of protrusions 32 is constant across the transfer layer 28. However, with reference to FIG. 3, it can be seen that the some of the apertures that would otherwise be present with the nested hexagon pattern were partly or fully obscured during the formation of the troughs 38 (or when the wire was wrapped around the forming screen). Accordingly, the troughs 38, which are not apertured, will result in a reduction in the open area that the transfer layer 28 might otherwise have without the troughs 38. Accordingly, to calculate the open area of the transfer layer 28, it is necessary to subtract the area occupied by the troughs 38 from the above open area calculations.

The area occupied by the troughs 38 is calculated based on the width of the trough 38 and the number of troughs 38 per lineal inch of film. For example, the transfer layer 28 identified as Example 1 below has a pattern of nested hexagons (as seen in FIG. 3) with an open area of 19.7%. The troughs 38 occupy 36.8% of that open area, such that the total open area of the film was reduced to 12.5%.

Transfer layers 28 having an open area as low a 2% have been tested with good results. It is unlikely that a transfer layer 28 having open area greater than 50% is practical for absorbent article applications. Thus, a broad range of open area for the transfer layers 28 is from about 2% to about 50%. Transfer layers 28 having an open area of 3% to 7% are useful, but the preferred range is from about 10% to about 20%. Open areas of about 30% to about 50% also function but are not generally preferred because of the reduced ability to transfer liquids outside of the immediate insult region declines as the open area increases above 30%.

The transfer layer 28 has a loft in the range of 1.651 mm to 0.3429 mm. Loft is defined as the total Z-dimension of the transfer layer 28, measured from secondary plane 44 or the external surface of the ridge 30 to the third plane 46 or the terminal end 48 of the protrusions 32 of the transfer layer 28.

A transfer layer 28 with a hexagonal pattern and an 8.75 mesh has a theoretical loft of 0.9398 mm for the protrusion only. In making the transfer layers 28 using a wire-wound forming screen as discussed above, approximately 30% of the wire is inset into a groove in the forming screen and 70% of the diameter of the wire is exposed above the surface of the forming screen and will translate to increased loft of the transfer layer 28. If a 1.1684 mm diameter wire were used, with 30% of the diameter buried in the groove in the screen, 0.8128 mm of the wire diameter would be protruding. By adding the exposed portion of the wire to the theoretical loft of the transfer layer 28 based on the diameter of the protrusions, the total theoretical loft of the transfer layer 28 would be 1.7526 mm. Generally speaking, a loft of 0.3429 mm or more is sufficient for preventing rewet in absorbent articles.

With reference to FIG. 5 in particular, when viewed from the female surface 40 of the transfer layer 28, the troughs 38 will appear as ridges 30. If a wire is used to create the troughs 38, as discussed above, the ridges 30 would have a rounded or semicircular appearance. These ridges 30 are positioned in close contact with the absorbent core 24 of the absorbent article 10 whereby the male surface 34 and apertures in the third plane 36 at the terminal end 48 of protrusions 32 are in close proximity to, or in contact with, the topsheet 22. The rounded or semicircular configuration of the ridges 30 is generally preferred because it facilitates movement of liquids along the troughs 38, similar to water moving in a gutter or storm culvert or drain pipe.

With particular reference to FIG. 5, the transfer layer 28 is seen having a generally sinusoidal appearance when viewed in cross-section. The sinusoidal appearance provides for an alternating series of peaks 50 and troughs 38, wherein the peaks 50 correspond to the areas of the transfer layer 28 between the troughs 38. The protrusions 32 are positioned atop the peaks 50 with the sidewalls 54 forming protruded extensions of the transfer layer 28, terminating in an aperture 36 on the male surface 34 of the transfer layer 28.

The transfer layers 28 can be made from thermoplastic polymeric materials conventionally used to make apertured formed films. For example, transfer layers 28 may comprise at least one polymer selected from polyolefins (e.g., C2-C10 olefins such as polyethylene, polypropylene, and copolymers); polyesters; plastomers; polyamides (e.g., nylon); polystyrenes; polyurethanes; vinyl polymers; acrylic and/or methacrylic polymers; elastomers (e.g., styrene block copolymer elastomers); polymers from natural renewable sources; biodegradable polymers; and mixtures or blends thereof. The thermoplastic material used to make transfer layer 28 preferably contains polyethylene having a density in the range of from 0.919 g/cc to 0.960 g/cc, with the more preferred range being from 0.930 g/cc to 0.950 g/cc. The general melt indices range for a typical material is preferably from 0.10 to 8.50 g/10 min., with the more preferred range typically being from 1.5 to 4.5 g/10 min. The gauge of the film can vary from 0.01778 mm to 0.127 mm. Gauge is the term used to define the loft of the transfer layer 28 without any protrusions 16 or troughs 38 and identifies the nominal thickness of the film. Basis weight of the film is defined as the weight of the film per unit area. Basis weight of the films can range from 16.8-120.5 gsm. Preferred ranges are from 21.7 gsm-72.3 gsm, most preferably 26.0-55.0 gsm. Additionally, any of a variety of additives may be added to the polymers and may provide certain desired characteristics, including, but not limited to, roughness, reduction of anti-static charge build-up, abrasion resistance, printability, write-ability, opacity, hydrophilicity, hydrophobicity, processibility, UV stabilization, color, etc. Such additives are well known in the industry and include, for example, calcium carbonate (abrasion resistance), titanium dioxide (color and opacity), silicon dioxide (roughness), surfactants (hydrophilicity/ hydrophobicity), process aids/plastomers (processibility), etc. The most preferred embodiment comprises 60% of high density polyethylene (density of 0.960 g/cc), 29% of liner grade low density polyethylene (density of 0.921 g/cc), 6% of a white pigment concentrate yielding 4.0% ash of inorganic titanium dioxide, and 5% of a surfactant concentrate yielding 6000ppm surfactant.

In use, the transfer layer 28 will be adhered to the absorbent core 24, the topsheet 22, or both, as mentioned above, with the external surface of the ridges 30 (secondary plane 44) positioned against the absorbent core 24. When an insult occurs, the bodily liquids will pass through the topsheet 22 and contact the male surface 34 of the transfer layer 28. Some of the fluids will enter the apertures in the third plane 46 and flow through the protrusions 32 to the absorbent core 24 under the influence of gravity. The majority of the liquids, however, will enter the troughs 38 where they will be distributed along the Y-direction of the article. As the troughs 38 fill up, the liquids begin to flow into the apertures in the third plane 36 and down through the hollow protrusions 32 to the absorbent core 24.

The topsheet 22 is on the body facing side of the absorbent article and typically comprises a liquid pervious material that allows liquid from an insult to transfer from the body-facing surface of the absorbent article to the absorbent core 24. The topsheet 22 is typically in close proximity or even direct contact with the wearer's skin during use and is typically made of a soft material such as a nonwoven fibrous material, an apertured film, or a combination of these materials made into a unitary composite. The topsheet 22 is typically designed to retain a comfortable, dry feel to the wearer even after an insult.

The backsheet 26 is positioned on the garment facing side or outside surface of the absorbent article. A backsheet 26 may be a liquid impervious film that does not allow liquid to transfer from within the absorbent article to the exterior surface of the absorbent article or to the garment of the wearer. It is also common for backsheet 26 to contain a liquid impermeable film laminated to a fibrous nonwoven web, which gives the film a textile or cloth-like appearance. A breathable backsheet is impervious to liquid, yet allows water vapor to pass out of the absorbent article. This lowers the humidity felt by the wearer and thereby increases the comfort to the wearer. Breathable backsheets may utilize an apertured film or a microporous breathable film, both of which are known in the art, and may also include a nonwoven fibrous web for improved aesthetics and consumer acceptance.

The absorbent core 24 absorbs the insult and retains the liquid while the absorbent article is in use. The absorbent core 24 should adequately absorb an insult or multiple insults and substantially retain the insult until the absorbent article is removed and discarded. The storage capacity of the absorbent core 24 and the efficiency of distribution of an insult across the absorbent core 24 determine the amount of liquid that may be held in the absorbent article. The absorbent material in an absorbent core 24 may comprise any liquid absorbent material such as, but not limited to, cellulose materials including fibers, cellular sponge or foam materials, super absorbent materials, such as superabsorbent polymers, hydrocolloidal materials, gel materials and combinations thereof. It is within the contemplated scope of the present disclosure that one or more of these types of absorbent materials are useful in specific embodiments. In particular, in certain embodiments, the absorbent material may comprise a mixture of absorbent granular materials and finely chopped cellulose fibers.

Particularly useful absorbent materials are high absorbency gel-type materials which are generally capable of absorbing about 10 to about 50 times their weight in fluid. As is generally known in the art, the rate at which the core absorbs liquids is inversely proportional to the ability of the core to hold the liquids absorbed. Thus, the superabsorbent materials used in cores are very good at holding liquids, but are relatively slow at liquid uptake. The delay in liquid uptake results in more unabsorbed or free fluid in the article, and thus decreases the rewet performance of the article. Because use of these materials has other benefits, such as reduced bulk of the core, the slower uptake is generally outweighed by the other advantages.

### EXAMPLES

A series of transfer layer films were made by extruding a molten polymer blend consisting of 60% of high density polyethylene (density of 0.960 g/cc), 29% of liner grade low density polyethylene (density of 0.921 g/cc), 6% of a white pigment concentrate yielding 4.0% ash of inorganic titanium dioxide, and 5% of a surfactant concentrate yielding 6000 ppm surfactant. The films were made by casting the molten polymer blend onto a forming screen and a then applying vacuum to form the apertures (36, 52) in accordance with well known vacuum aperturing processes.

The forming screen used to make the example transfer layers had a groove cut into it and a wire wrapped in the groove as described above. For the control film, the screen was used without cutting a groove and with no wire. The aperture pattern of the screen, the Threads per Inch of the grove, the diameter of the wire used, and the loft of the resulting film are all reported in Table 1. All of the films had a nominal basis weight of 36.8 gsm. The nominal gauge of the films, which is the thickness of the film without any apertures (36, 52) or troughs 38, is 0.0381mm.

TABLE 1

| | | Transfer | Forming | | |
|---|---|---|---|---|---|
| | | Layer | screen wire | | |
| Sample | Transfer | aperture | diameter, | Forming | Loft, |
| No. | Layer mesh | pattern | mm | screen TPI | mm |
| Control 1 | n/a | n/a | n/a | n/a | n/a |
| Control 2 | 8.75 | hexagonal | n/a | n/a | 1.14 |
| | | pattern | | | |
| EX 1 | 8.75 | hexagonal | 1.168 | 8 | 1.10 |
| | | pattern | | | |
| EX 2 | 8.75 | hexagonal | 1.168 | 6 | 0.96 |
| | | pattern | | | |
| EX 3 | 8.75 | hexagonal | 0.584 | 6 | 1.11 |
| | | pattern | | | |
| EX 4 | 40 | hexagonal | 0.305 | 20 | 0.64 |
| | | pattern | | | |

The film samples of Table 1 were then tested for fluid distribution in the X-direction and Y-direction in a size 6 Pampers® Baby Dry diaper available from Procter & Gamble. The leg cuffs of the diaper were cut off so that the diaper would lay flat. A cross-sectional view of the diaper 64, as seen in FIG. 6, comprised a nonwoven topsheet 66, a sub-layer 68, absorbent core 70, a layer of super absorbent particles 72, and a fluid barrier backsheet 74. This diaper 64 was used as the control, Identified as Control 1 test specimen 76.

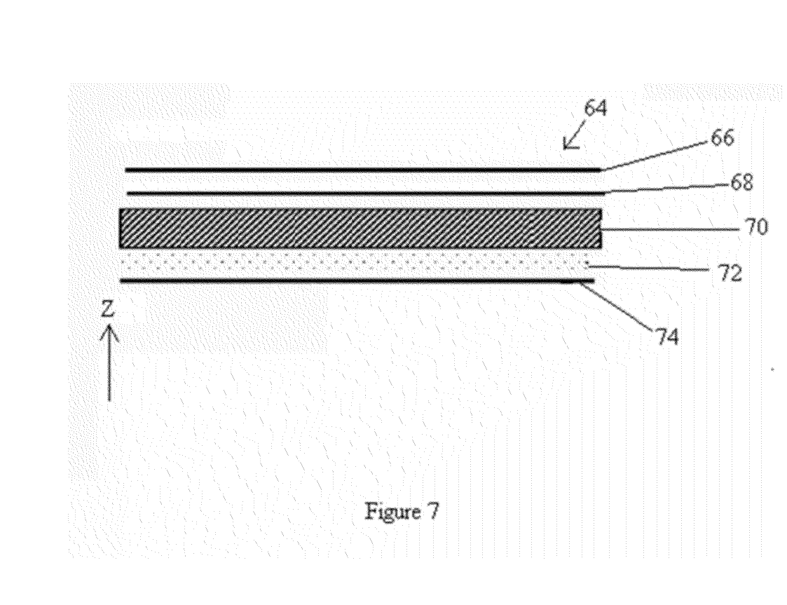

For the exemplified transfer layers, the transfer layers 28 were cut to a size of 76.2 mm wide by 152.4 mm long and placed in the diaper 64 between the topsheet 66 and the sub-layer 68 as seen in FIG. 7, with the male surface 34 of the transfer layer 28 oriented toward the nonwoven topsheet 66 to form a test specimen 76.

For comparison, a commercially available apertured film, having an 8.75 mesh, nested hexagon aperture pattern and sold by Tredegar Film Products Corporation under the brand AquiDry™ Classic, was used in the diaper 64 with the female side oriented toward the topsheet 66 and the male side oriented toward the sub-layer 68 to form a test specimen 76. This is identified in the data as Control 2.

The apparatus used to test fluid distribution and the test specimen 76 is illustrated in FIGS. 8 and 9. The apparatus 78, as seen in FIG. 8, is placed on a support surface 80. The apparatus 78 has a plate 82. The plate 82 is approximately 152.4 mm wide by 381 mm long, having an elevated plate edge 84, a lowered plate edge 86, a left plate edge 88 and a right plate edge 90. The plate 82 is fixed at an angle θ of 8.5°. Clamps 92 are provided near the elevated plate edge 84 and the lowered plate edge 86 to hold a test specimen 76 at the front side edge 18 and the back side edge 20 of the test specimen 76 in a fixed position during the test. Electrical probes (Probe #1 94, Probe #2 96, Probe #3 98 and Probe #4 100) are provided at spaced-apart locations along the length of the plate 82. The probes (94, 96, 98, 100) protrude a distance of 7.94 mm above the surface of plate 82 to penetrate into the absorbent core 24 of the test specimen 76.

The test specimens 76 were clamped onto plate 82 using clamps 92 with the backsheet 74 placed adjacent to the plate 82 and the topsheet 66 facing upwards. The test specimen 76 was oriented on the plate 82 such that the front side edge 18 of the test specimen 76 (diaper 64) was located at the elevated plate edge 84 and the back side edge 20 of the test specimen 76 (diaper 64) is located at the lowered plate edge 86.

The apparatus 78 used for the test contains 4 probes (Probe #1 94, Probe #2 96, Probe #3 98 and Probe #4 100) all of which were connected to a timing device (not shown). Probes #1 and #2 (94, 96) are located in close proximity to one another and both within the insult area defined by the splash ring 102, which comprised a 12.7 mm long piece of PVC pipe having an internal diameter of 38.1 mm. Probe #3 98 was located near the lowered plate edge 86 and the back side edge 20 test specimen 76. Probe #4 100 is located on the right plate edge 90 and the left side edge 14 of the test specimen 76. In placing the test specimens 76 in the apparatus, the transfer layer 28 was positioned 25.4 mm above the Probe #3 98.

After the test specimen is clamped in place on the plate 82, the splash ring 102 is placed around Probes #1 and #2. 50 ml of a 0.9% Isotonic Saline Solution (Ricca Chemicals, Catalog No. 7210-5) is then applied using a pipette to the center of splash ring 102 in two 25 ml aliquots, separated from one another by a 10 second delay. As the saline solution is introduced, the splash ring 102 will contain the solution in the target area in the event of any pooling of solution on the topsheet 66.

After the liquid insult is applied, an electrical connection forms between Probes #1 and #2 when the liquid insult contacts both Probes #1 and #2, and the timing device is activated. When the saline solution reaches Probe #3, the timing device will record the time lapse. This represents the time needed for the liquid to flow in the X-direction. Similarly, when the saline solution reaches Probe #4, the timing device will also record that time lapse. This time lapse represents the time need for the liquid to flow in the Y-direction. The test was repeated four times and the lapse times were averaged. Results are reported in Table 2.

TABLE 2

| | X-direction to | Y-direction to |
|---|---|---|
| | Probe #3 | Probe #4 (average |
| | (average sec) | sec) |
| Control 1 | 5.0 | 140.3 |
| Control 2 | 6.3 | 47.3 |
| EX 1 | 13.7 | 8.0 |
| EX 2 | 11.7 | 9.7 |
| EX 3 | 15.3 | 6.3 |
| EX 4 | 26.7 | 10.0 |

These data demonstrate that absorbent articles using the transfer layers 28 with troughs 38 and the male side 34 oriented toward the topsheet 22 significantly reduce the flow of liquids in the X-direction and significantly increase the flow of liquids in the Y-direction (slower times in the X-direction and faster times in the Y-direction). It is believed that the increase time needed for the liquid to reach the Probe #3 98 indicates that the liquids would be significantly less likely to reach the leg cuff area, thus offering improved protection against leakage. It is also believed that a faster distribution speed in the Y direction also provides for more uniform distribution of liquid throughout the absorbent article.

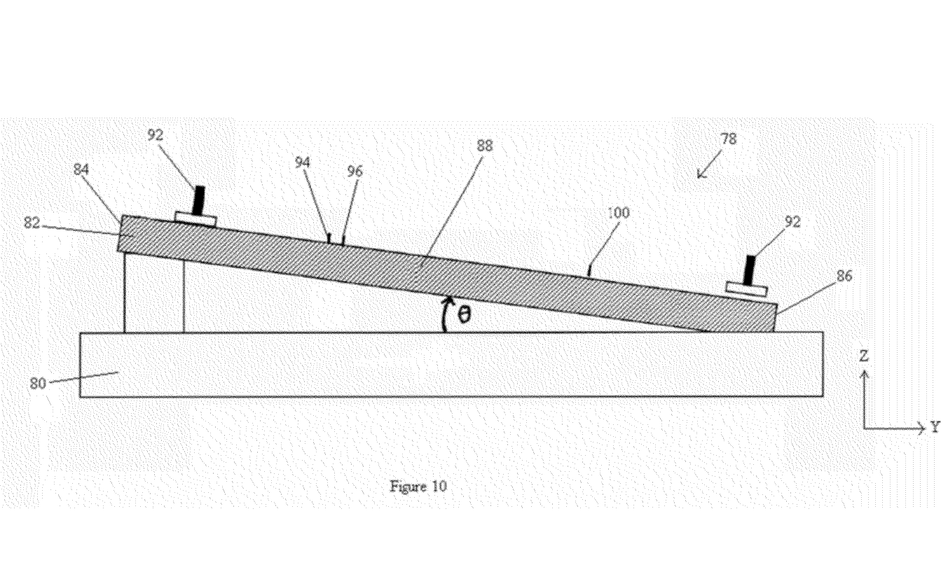

With reference to FIG. 10, after completing the test to determine the time to reach Probe #3 and Probe #4, the test specimens 76 were cut at cut lines 104 into 4 segments (identified as segments S-1, S-2, S-3 and S-4 in FIG. 10) with each segment being approximately 50.8 mm long in the Y-direction. Each of the segments, S-1 to S-4, was weighed. The difference in weight provides an indication of the distribution of liquids throughout the test specimen 76 and the ability of the test specimen 76 to move fluids from the original insult area to other areas of the absorbent core 24. It is believed that the greater the uniformity of distribution, the more efficient the article will be at absorbing and holding fluids, making the article less prone to leakage.

TABLE 4

| Segment | Control 1 | Control 2 | EX 1 | EX 2 | EX 3 | EX-4 |
|---|---|---|---|---|---|---|
| S-1 | 14.8 | 17.1 | 11.1 | 10.5 | 11.3 | 11.6 |
| S-2 | 27.0 | 25.5 | 19.3 | 22.8 | 18.6 | 22.4 |
| S-3 | 16.9 | 17.2 | 12.6 | 17.6 | 17.2 | 16.0 |
| S-4 | 5.2 | 5.3 | 16.8 | 12.4 | 16.9 | 14.3 |

In order to demonstrate the improvements seen when using the transfer layer 28 of the present application (comprising troughs 38 and oriented with the third plane 46 toward the topsheet 22), as opposed to the more conventional orientation with the third plane 46 toward the absorbent core shown in U.S. Pat. No. 7,378,568, the following test was conducted.

A 137.9 mm×304.8 mm piece of absorbent filter paper (#989, Empirical Manufacturing Company) was placed on a 10° inclined surface. To generate the date for Control 1, no film was used for the test specimen. For Control 2, the AquiDry™ Classic film was used with the absorbent filter paper for the test specimen. For Examples 1-3, the transfer layers 28 EX 1 to EX 3 identified in Table 1 were used with the absorbent filter paper for the test specimen, respectively. The test specimens had both orientations of the transfer layer 28, the transfer layer 28 was placed over the filter paper with the third plane 46 (or the male side of the comparative example) oriented up such that the secondary plane 44 (or the female side of the comparative example) of the transfer layer 28 was in contact with the paper or with the third plane 46 (or the male side of the comparative example), such that the male surface/side was in contact with the paper.

A 25 ml volume of 0.9% isotonic sodium chloride solution at 70 dynes (mN/m) then applied to the film within 5 seconds. The point of insult was the center of the test specimen 76 in the X-direction, and 25.4 mm from the top edge of the transfer layer 28 or the comparative film. The distance between the insult point and the edge of the transfer layer 28 or the comparative film in the Y-direction at the bottom of the 10° inclined surface was thus 279.4 mm. The distance that the saline solution travelled from the insult point before being fully absorbed was then measured. Results are reported in Table 3.

TABLE 3

| | Male Side Down | Male Side Up |
|---|---|---|
| | Distance, cm | Distance, cm |
| Control 1 | 12.1 | 12.1 cm |
| | (no film) | (no film) |
| Control 2 | 13.2 | 13.6 |
| EX 1 | 17.0 | 19.3 |
| EX 2 | 17.0 | 17.6 |
| EX 3 | 14.4 | 15.8 |

As can be seen in the data in Table 3, it is believed that with the male surface 34 of the transfer layer 28 oriented toward the topsheet 22, the transfer layer 28 permits greater distribution of the liquids in the Y-direction as compared to the same transfer layer 28 oriented with the male surface 34 toward the absorbent core 24.

Every document cited herein, including any cross referenced or related patent or application, is hereby incorporated herein by reference in its entirety unless expressly or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

It is to be understood that although this disclosure describes several embodiments, various modifications apparent to those skilled in the art may be made without departing from the invention as described in the specification and claims herein.

We claim:

1. An absorbent article comprising a width and a length, the length running from a front side edge of the article, through a crotch area, to a back side edge of the article, the width of the article is perpendicular to the length and runs from a left side edge of the article to the right side edge of the article; the absorbent article further comprises a topsheet, a backsheet, an absorbent core positioned between the topsheet and the backsheet, and a transfer layer positioned between the topsheet and the backsheet in the crotch area; the transfer layer comprises a primary plane, a secondary plane and a third plane; extended from primary plane to the secondary plane are a plurality of troughs; extending from primary plane to the third plane are a plurality of protrusions; the protrusions comprise an apertures in the primary plane, sidewalls and a terminal end comprising an aperture, the terminal end and the aperture are located in the third plane; wherein the third plane is in contact with the topsheet and the plurality of ridges are orientated parallel to the length of the absorbent article.

2. The article of claim 1, wherein the secondary plane is positioned in close contact with the absorbent core.

3. The article of claim 1, wherein the troughs comprise a semicircular cross-sectional configuration.

4. The article of claim 1, wherein the sidewalls taper inward such that the aperture on the primary plane is larger than the aperture on the third plane.

5. The article of claim 1, wherein the protrusions are arranged in a 60° equilateral triangular array.

6. The article of claim 1, wherein the transfer layer has a total open area of 2% to 50%, and more preferably between 10% and 20%.

7. The article of claim 1, wherein the transfer layer has a loft of at least 0.3429 mm.

8. The article of claim 1, wherein the transfer layer has a sinusoidal shape in cross-section and comprises alternating peaks and troughs, wherein the protrusions are located at an apex of the peaks.

9. The article of claim 1, wherein the topsheet is selected from the group consisting of nonwoven fibrous webs, apertured films, and combinations thereof.

10. The article of claim 1, wherein the transfer layer comprises a vacuum formed apertured film, the plurality of hollow, tapered protrusions originating on the primary plane and tapering toward an aperture at a terminal end of the protrusion in the third plane, said plurality of protrusions being arranged in a 60° equilateral triangular array, wherein the troughs have a longitudinal axis oriented in the Y-direction of the article, said troughs being arranged in spaced apart parallel rows and alternating with rows of said protrusions.

11. The article of claim 9, wherein the troughs comprise troughs with semicircular cross-sections, and wherein said troughs are positioned in close contact with the absorbent core.

12. A cylindrical vacuum forming screen comprising a base pattern of apertures and one or more shallow spiral grooves encompassing one or more wires affixed around the circumference of the forming screen, wherein the plurality of shallow spiral grooves are present in the base pattern with a thread per inch patter of from 20 to 6, the shallow spiral grooves encompassing one or more wires, the wire having a diameter from 0.305 mm to 2.362.
